(19) United States
(12) Patent Application Publication
(10) Pub. No.: US 2026/0294320 A1
(43) Pub. Date: Oct. 1, 2026

(54) DETECTION OF CHANGES IN CARDIAC HEALTH

(71) Applicant: AliveCor, Inc., Mountain View, CA (US)

(72) Inventor: Trisha Dwivedi, San Jose (CA)

(21) Appl. No.: 19/632,086

(22) Filed: Mar. 27, 2026

Related U.S. Application Data
(60) Provisional application No. 63/781,190, filed on Mar. 31, 2025.

(51) Int. Cl. A61B 5/366; A61B 5/355; A61B 5/358

(52) U.S. Cl. CPC A61B 5/366; A61B 5/355; A61B 5/358

(57) ABSTRACT

Techniques for longitudinally analyzing a user's ECG data to identify changes in the user's ECG data that could indicate the onset of cardiac conditions are disclosed. In response to receiving a new electrocardiogram (ECG) data entry comprising new ECG data, a cross-correlation of the new ECG data with a baseline median beat of the user is determined. Whether the new ECG data of the user represents a deviation from the baseline median beat with respect to characteristics that indicate abnormal morphologies is determined based on the determined cross-correlation. In response to determining that the new ECG data represents a deviation with respect to the one or more characteristics, a deviation indication is appended to the new ECG data entry. After a threshold number of new ECG data entries have a deviation indication appended to them, generating an alert indicating physician analysis of the user's ECG data is required.

### CROSS-REFERENCE TO RELATED APPLICATIONS

This application claims priority to U.S. Provisional Application No. 63/781,190, filed Mar. 31, 2025 and entitled “DETECTION OF CHANGES IN CARDIAC HEALTH,” the contents of which are hereby incorporated by reference.

### TECHNICAL FIELD

Aspects of the present disclosure relate to monitoring of cardiac health, and more specifically to monitoring of deviations in electrocardiogram (ECG) data that are indicative of cardiac health trends that implicate cardiac conditions.

### BACKGROUND

Cardiovascular diseases are the leading cause of death in the world. In 2008, 30% of all global death can be attributed to cardiovascular diseases. It is also estimated that by 2030, over 23 million people will die from cardiovascular diseases annually. Cardiovascular diseases are prevalent across populations of first and third world countries alike, and affect people regardless of socioeconomic status.

Arrhythmia is a cardiac condition in which the electrical activity of the heart is irregular or is faster (tachycardia) or slower (bradycardia) than normal. Although many arrhythmias are not life-threatening, some can cause cardiac arrest and even sudden cardiac death. Indeed, cardiac arrhythmias are one of the most common causes of death when travelling to a hospital. Atrial fibrillation (A-fib) is the most common cardiac arrhythmia. In A-fib, electrical conduction through the ventricles of heart is irregular and disorganized. While A-fib may cause no symptoms, it is often associated with palpitations, shortness of breath, fainting, chest pain or congestive heart failure and also increases the risk of stroke. A-fib is usually diagnosed by taking an electrocardiogram (ECG) of a subject. To treat A-fib, a patient may take medications to slow heart rate or modify the rhythm of the heart. Patients may also take anticoagulants to prevent stroke or may even undergo surgical intervention including cardiac ablation to treat A-fib. In another example, an ECG may provide decision support for Acute Coronary Syndromes (ACS) or other cardiac conditions by interpreting various rhythm and morphology conditions, including Myocardial Infarction (MI) and Ischemia.

Often, a patient with A-fib (or other type of arrhythmia) is monitored for extended periods of time to manage the disease. For example, a patient may be provided with a Holter monitor or other ambulatory electrocardiography device to continuously monitor the electrical activity of the cardiovascular system for e.g., at least 24 hours. Such monitoring can be critical in detecting conditions such as acute coronary syndrome (ACS), among others.

The American Heart Association and the European Society of Cardiology recommends that a 12-lead ECG should be acquired as early as possible for patients with possible ACS or other cardiac conditions when symptoms present. Prehospital ECG has been found to significantly reduce time-to-treatment and shows better survival rates. The time-to-first-ECG is so vital that it is a quality and performance metric monitored by several regulatory bodies. According to the national health statistics for 2015, over 7 million people visited the emergency department (ED) in the United States (U.S.) with the primary complaint of chest pain or related symptoms of cardiac conditions. In the US, ED visits are increasing at a rate of or 3.2% annually and outside the U.S. ED visits are increasing at 3% to 7%, annually.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1A is a diagram that illustrates example electrocardiogram (ECG) readings, in accordance with some embodiments of the present disclosure.

FIG. 1B is a block diagram that illustrates example electrocardiogram (ECG) readings, in accordance with some embodiments of the present disclosure.

FIG. 2 is a block diagram that illustrates an example system, in accordance with some embodiments of the present disclosure.

FIG. 3 is a block diagram that illustrates an example cloud storage system, in accordance with some embodiments of the present disclosure.

FIGS. 4A-4D are diagrams illustrating ST segment and QRS complex cross-correlation between a baseline median beat and new ECG data of a user, in accordance with some embodiments of the present disclosure.

FIG. 4E is a diagram illustrating some of the information included in an ECG deviation report, in accordance with some embodiments of the present disclosure.

FIG. 5 is a flow diagram of a method for longitudinally analyzing a user's ECG data to identify changes in the user's ECG data that could indicate the onset of cardiac conditions, in accordance with some embodiments of the present disclosure.

FIG. 6 is a block diagram of an example computing device that may perform one or more of the operations described herein, in accordance with some embodiments of the present disclosure.

### DETAILED DESCRIPTION

In the following detailed description of embodiments of the present disclosure, numerous specific details are set forth in order to provide a more thorough understanding of the disclosure. However, it will be apparent to one of ordinary skill in the art that the concepts within the disclosure can be practiced without these specific details. In other instances, well-known features have not been described in detail to avoid unnecessarily complicating the description.

ECG monitoring devices can provide detailed ECG measurement and visualization at the point of assessment. However, such information is often useful only to users who have existing cardiac conditions (and/or are currently experiencing symptoms of a cardiac condition) and may be of less benefit to users with no or limited heart-related issues or concerns. In addition, such devices do not track and analyze a user's ECG data over time, which can help users with and without diagnosed conditions understand when their heart is trending towards a point where they may need additional clinical support.

Many users of such ECG monitoring devices are participants in a corresponding subscription service where ECG measurements may be taken at predefined intervals and/or in response to certain cardiac events and then stored e.g., in a database or in the cloud. Thus, users of such ECG monitoring devices often have years'worth of ECG data which can be leveraged to perform longitudinal monitoring of a user's data over time to detect changes that can help users with and without diagnosed conditions understand trends in their heart health and identify certain trends that may be indicative of a future cardiac condition(s).

Embodiments of the present disclosure address the above and other problems by providing techniques for longitudinally analyzing a user's ECG data to identify changes in the user's ECG data that could indicate the onset of cardiac conditions are disclosed. In response to receiving a new ECG data entry comprising new electrocardiogram (ECG) data, a cross-correlation of the new ECG data with a baseline median beat of the user is determined. Whether the new ECG data of the user represents a deviation from the baseline median beat with respect to one or more characteristics that indicate abnormal morphologies is determined based on the determined cross-correlation. In response to determining that the new ECG data represents a deviation with respect to the one or more characteristics, a deviation indication is appended to the new ECG data entry. After a threshold number of new ECG data entries have a deviation indication appended to them, an alert indicating physician analysis of the user's ECG data is required is generated.

An electrocardiogram (ECG) provides a number of ECG waveforms that represent the electrical activity of a person's heart. An ECG monitoring device may comprise a set of electrodes for recording these ECG waveforms (also referred to herein as “taking an ECG”) of the patient's heart. The set of electrodes may be placed on the skin of the patient in multiple locations and the electrical signal (ECG waveform) recorded between each electrode pair in the set of electrodes may be referred to as a lead. Varying numbers of leads can be used to take an ECG, and different numbers and combinations of electrodes can be used to form the various leads. Example numbers of leads used for taking ECGs are 1, 2, 3, 6, and 12 leads.

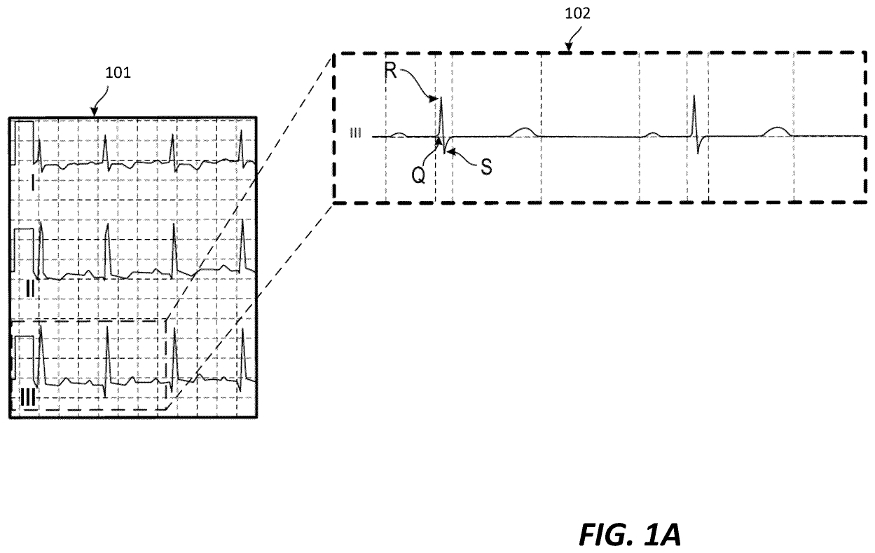

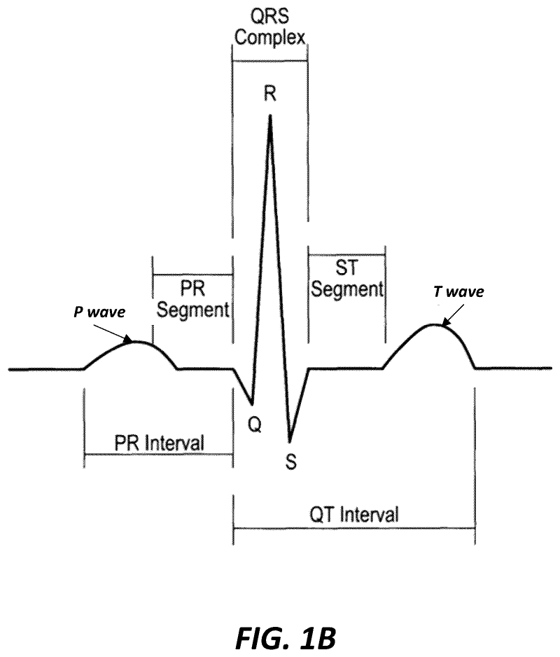

The ECG waveform obtained from each different electrode pair/lead may be different/unique (e.g., may have different morphologies/amplitudes). This is because although the various leads may analyze the same electrical events, each one may do so from a different angle. FIG. 1A illustrates a view 101 of an ECG waveform detected by each of 3 leads (I, II, and III) when a 3-lead ECG is taken as well as an exploded view 102 of the ECG waveform measured by lead III illustrating the QRS complex. As shown, the amplitudes and morphologies of the ECG waveform taken from leads I-III are all different, with the ECG waveform measured by lead III having the largest amplitude and the ECG waveform measured by lead I having the smallest amplitude.

There are different “standard” configurations for electrode placement that can be used to place electrodes on the patient. For example, an electrode placed on the right arm can be referred to as RA. The electrode placed on the left arm can be referred to as LA. The RA and LA electrodes may be placed at the same location on the left and right arms, preferably near the wrist in some embodiments. The leg electrodes can be referred to as RL for the right leg and LL for the left leg. The RL and LL electrodes may be placed on the same location for the left and right legs, preferably near the ankle in some embodiments. Lead I is typically the voltage between the left arm (LA) and right arm (RA), e.g. I=LA-RA. Lead II is typically the voltage between the left leg (LL) and right arm (RA), e.g. II=LL-RA. Lead III is the typically voltage between the left leg (LL) and left arm (LA), e.g. III=LL-LA. Augmented limb leads can also be determined from RA, RL, LL, and LA. The augmented vector right (aVR) lead is equal to RA−(LA+LL)/2 or −(I+II)/2. The augmented vector left (aVL) lead is equal to LA−(RA+LL)/2 or I−II/2. The augmented vector foot (aVF) lead is equal to LL−(RA+LA)/2 or II−I/2.

The ECG waveforms (each one corresponding to a lead of the ECG) recorded by the ECG monitoring device may comprise data corresponding to the electrical activity of the person's heart. A typical heartbeat may include several variations of electrical potential, which may be classified into waves and complexes, including a P wave, a QRS complex, a T wave, and a U wave among others, as is known in the art. Stated differently, each ECG waveform may include a P wave, a QRS complex, a T wave, and a U wave among others, as is known in the art. The shape and duration of these waves may be related to various characteristics of the person's heart such as the size of the person's atrium (e.g., indicating atrial enlargement) and can be a first source of heartbeat characteristics unique to a user.

For example, the QRS complex can correspond to the depolarization of the heart ventricles, and can be separated into three distinct waves—a Q wave, a R wave and a S wave. The duration, amplitude, and morphology of the QRS complex and each of the Q, R and S waves can vary in different individuals, and in particular can vary significantly for users having cardiac diseases or cardiac irregularities. For example, a Q wave that is greater than ⅓ of the height of the R wave, or greater than 40 ms (milliseconds) in duration can be indicative of a myocardial infarction and provide a unique characteristic of the user's heart. Similarly, other healthy ratios of Q and R waves can be used to distinguish different users' heartbeats. The duration of the QRS complex of a healthy heart can be in the range of 60 to 100 milliseconds (ms), but can vary due to abnormalities of conduction and thus can serve as another source of heartbeat characteristics unique to a user.

The electrical activity of the user US's heart can also include one or more characteristic durations or intervals that can be used to distinguish different users. For example, the electrical activity of the heart may include PR intervals and ST segments as known in the art. A PR interval can be measured from the beginning of the P wave to the beginning of the QRS complex. A PR interval can typically last 120 to 200 ms. A PR interval having a different duration can indicate one or more defects in the heart, such as a first degree heart block (e.g., a PR interval lasting more than 200 ms), a pre-excitation syndrome via an accessory pathway that leads to early activation of the ventricles (e.g., a PR interval lasting less than 120 ms), or another type of heart block (e.g., a PR interval that is variable). An ST segment can be measured from a QRS complex to a T wave, for example starting at the junction between the QRS complex and the ST segment and ending at the beginning of the T wave. An ST segment can typically last from 80 to 120 ms, and normally has a slight upward concavity. The combination of the length of ST segment, and the concavity or elevation of ST segment can also be used to generate characteristic information unique to each user's heartbeat.

FIG. 1B illustrates an example Lead I annotated to show the P, QRS, and T waves/complexes. Typically, an ECG of a normal beating heart has a predictable wave-form in each of the twelve ECG leads. ECG portions between two waves are referred to as segments and ECG portions between more than two waves are referred to as intervals. For example, the ECG portion between the end of the S wave (part of QRS complex) and the beginning of the T wave is referred to as the ST segment while the portion of the ECG between the beginning of the Q wave (part of QRS complex) and the end of the T wave is referred to as the QT interval.

The ECG waveforms may be analyzed (typically after standard filtering and “cleaning” of the signals) for various indicators that are useful in detecting cardiac events or status, such as cardiac arrhythmia detection and characterization. Such indicators may include ECG waveform amplitude and morphology (e.g., QRS complex amplitude and morphology), R wave-ST segment and T wave amplitude analysis, and heart rate variability (HRV), for example.

It should be noted that a set of two or more leads may be transformed to generate a full, 12-lead ECG. Such transformation may be performed using a machine learning model (e.g., a neural network, deep-learning techniques, etc.). The machine learning model may be trained using 12-lead ECG data corresponding to a population of individuals. The data, before being input into the machine learning model, may be pre-processed to filter the data in a manner suitable for the application. For example, data may be categorized according to height, gender, weight, nationality, etc. before being used to train one or more machine learning models, such that the resulting one or models are finely-tuned the specific types of individuals. In a further embodiment, the machine learning model may be further trained based on a user's own ECG data, to fine-tune and personalize the model even further to decrease any residual synthesis error.

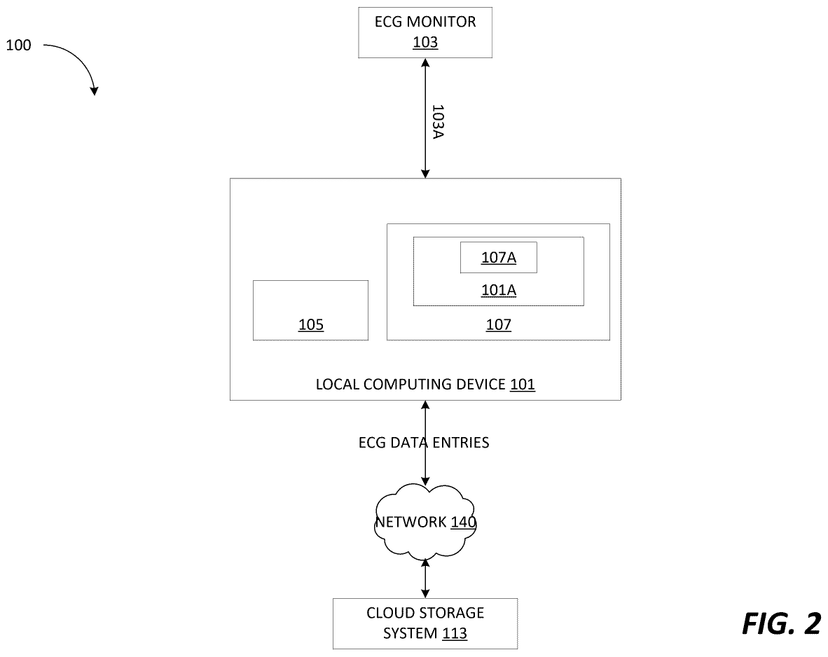

FIG. 2 illustrates a system 100 for longitudinally analyzing a user's ECG data to identify changes in the user's ECG data that could indicate the onset of cardiac conditions. The system 100 may comprise a local computing device 101 of the first user. The local computing device 101 may be a computing device that includes hardware such as a processing device 105 (e.g., processors, central processing units (CPUs)), memory 107 (e.g., random access memory (RAM)), storage devices (e.g., hard-disk drive (HDD), solid-state drive (SSD), etc.), and other hardware devices (e.g., sound card, video card, etc.). In some embodiments, memory 107 may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. Memory 107 may be configured for long-term storage of data and may retain data between power on/off cycles of the local computing device 101. The memory 107 may store the user data (discussed in further detail hereinbelow) accumulated over time for the user. The user data accumulated over time for the user may form a time series health record for the user.

The memory 107 may include an interface module 101A, which may enable the local computing device 101 to interface with the other components of the system 100. The interface module 101A may be configured to interface with one or more biometric sensors (e.g., ECG monitor 103) and may comprise software and a user interface for managing biometric data collected by the local computing device 101 from the one or more biometric sensors. The local computing device 101 may comprise any appropriate computing device, such as a tablet computer, a smartphone, a server computer, a desktop computer, a laptop computer, or a body-worn computing device (e.g., a smart watch or other wearable), for example. In some embodiments, the local computing device 101 may comprise a single computing device or may include multiple interconnected computing devices (e.g., multiple servers configured in a cluster).

The system 100 may further comprise one or more biometric sensors, such as the ECG monitor 103. The ECG monitor 103 may comprise a set of electrodes for recording ECG (electrocardiogram) data (also referred to herein as “taking an ECG”) of the user's heart. The ECG data can be recorded or taken using the set of electrodes which are placed on the skin of the first user in multiple locations as discussed hereinabove.

In some embodiments, the ECG monitor 103 may comprise a handheld ECG monitor (such as the KardiaMobile® or KardiaMobile® 6L device from AliveCor® Inc., for example) comprising a smaller number of electrodes (e.g., 2 or 3 electrodes). In these embodiments, the electrodes can be used to measure a subset of leads such as lead I (e.g., the voltage between the left arm and right arm) contemporaneously with lead II (e.g., the voltage between the left leg and right arm), and lead I contemporaneously with lead V2 or another one of the chest leads such as V5. It should be noted that any other combination of leads is possible. If desired, additional leads can then be algorithmically derived (e.g., by the ECG monitor 103 itself or the local computing device 101) from the determined subset of leads. For example, augmented limb leads can also be determined from the values measured by the LA, RA, LL, and RL electrodes. The augmented vector right (aVR) may be equal to RA−(LA+LL)/2 or −(I+II)/2. The augmented vector left (aVL) may be equal to LA−(RA+LL)/2 or I−II/2. The augmented vector foot (aVF) may be equal to LL−(RA+LA)/2 or II-I/2. In some embodiments, the ECG monitor 103 itself or the local computing device 101 may utilize a machine learning (ML) model to derive the full 12 lead set from a measured subset of leads. In some embodiments, the ECG monitor 103 may be in the form of a smartphone, or a wearable device such as a smart watch. In some embodiments, the ECG monitor 103 may be a handheld sensor coupled to the local computing device 101 with an intermediate protective case/adapter.

The ECG monitor 103 may be used by a first user to perform an ECG and obtain ECG data (where the ECG data comprises a waveform for each lead the ECG is being performed with) and transmit the measured ECG data to the local computing device 101 by connection 103A as described in further detail herein. The connection 103A may comprise a wired or wireless connection (e.g., a Wi-Fi connection, a Bluetooth® connection, a near-field communication (NFC) connection, an ultrasound signal transmission connection, etc.).

ECG data may be continually recorded by the user at regular intervals. For example, the interval may be once a day, once a week, once a month, or some other predetermined interval. Alternatively, or additionally, ECG data can be recorded on demand by the user at various discrete times, such as when the user feels chest pains or experiences other unusual or abnormal feelings, or in response to an instruction to do so from e.g., the user's physician. In another embodiment, ECG data may be continuously recorded over a period of time (e.g., by a Holter monitor or by some other wearable device).

The interface module 101A may timestamp the ECG data from each recording and store the timestamped ECG data as an ECG data entry in the memory 107 of the local computing device 101. In some embodiments, the ECG data from each recording may be annotated with additional data by the user or health care provider to describe user characteristics. For example, the local computing device 101 (e.g., the interface module 101A thereof) may include a user interface for data entry that allows the user to enter their user characteristics. Examples of user characteristics may include age, sex, race, ethnicity, relevant medical history, location, diet (e.g., food/drink habits), medication/drug consumption, exercise patterns, sleep/rest patterns, feelings of stress, anxiety, pain or other unusual or abnormal feelings, activities performed before, during, or after the ECG data recording, or any other user specific circumstance or factors that may affect the user's ECG data. The local computing device 101 may append the user characteristics to the timestamped ECG data and store the timestamped ECG data and user characteristics as an ECG data entry in the memory 107 for further analysis (e.g., ECG interpretation). Alternatively, or in addition to, the local computing device 101 may transmit the ECG data entry to the cloud storage system 113. Because the ECG data entry is time stamped and includes user characteristics, the ECG data can be matched or correlated with an activity or circumstance of interest. As described in further detail herein, this also allows for comparison of the ECG data before, after and during the activity or circumstance of interest so that the effect on the ECG data can be determined and accounted for during further analysis.

Each ECG data entry can be transmitted by the local computing device 101 to the cloud storage system 113 for storage and analysis. The transmission can be real-time, at regular intervals such as hourly, daily, weekly and/or any interval in between, or can be on demand. The local computing device 101 and the cloud storage system 113 may be coupled to each other (e.g., may be operatively coupled, communicatively coupled, may communicate data/messages with each other) via network 140. Network 140 may be a public network (e.g., the internet), a private network (e.g., a local area network (LAN) or wide area network (WAN)), or a combination thereof. In one embodiment, network 140 may include a wired or a wireless infrastructure, which may be provided by one or more wireless communications systems, such as a Wi-Fi hotspot connected with the network 140 and/or a wireless carrier system that can be implemented using various data processing equipment, communication towers (e.g., cell towers), etc. The network 140 may carry communications (e.g., data, message, packets, frames, etc.) between the local computing device 101 and the cloud storage system 113.

The ECG data entries accumulated over time for a particular user may form a time series health record for that particular user. The cloud storage system 113 may comprise any suitable type of computing device or machine that has a programmable processor including, for example, a server computer, a desktop computer, laptop computer, tablet computer, smartphone, etc. In some embodiments, the cloud storage system 113 may comprise a single computing device or may include multiple interconnected computing devices (e.g., multiple servers configured in a cloud storage cluster).

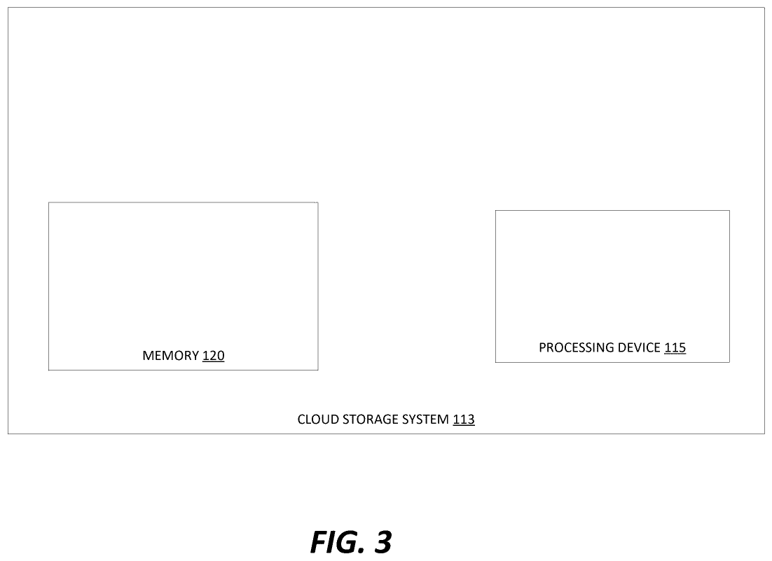

FIG. 3 illustrates the cloud storage system 113 in accordance with some embodiments of the present disclosure. As shown in FIG. 3, the cloud storage system 113 may be a computing device that includes hardware such as processing device 115 (e.g., processors, central processing units (CPUs)), memory 120 (e.g., random access memory (RAM)), storage devices (e.g., hard-disk drive (HDD), solid-state drive (SSD), etc.), and other hardware devices (e.g., sound card, video card, etc.). In some embodiments, memory 120 may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. Memory 120 may be configured for long-term storage of data and may retain data between power on/off cycles of the cloud storage system 113. The memory 120 may store the user data accumulated over time for the user as well as a multitude of other users. The user data accumulated over time for a particular user may form a time series health record for that particular user.

The cloud storage system 113 may provide third parties access to the user's ECG data entries. Examples of such third parties may include the user's physician, cardiac technicians, other cardiac specialists, and system administrators and managers. The third party may access the cloud storage system 113 via any appropriate type of computing device (e.g., personal computer, tablet computer, or smartphone) through network 140.

The interface module 101A may further include longitudinal monitoring logic 107A that is configured to perform longitudinal monitoring of the user's ECG data entries in order to identify changes in the user's ECG data that could indicate the onset of cardiac conditions. In this way, the longitudinal monitoring logic 107A may help users with and without diagnosed conditions understand when their heart is trending towards a point where they may need additional clinical support.

The T-wave inversion and the ST depression on the lateral leads of an ECG are two ECG characteristics that can indicate abnormal morphologies (e.g., ischemia and hypertrophy). For example, it has been shown that the T-wave inversion in lead I of an ECG is highly correlated with abnormal morphologies. Thus, the longitudinal monitoring logic 107A may monitor for deviations from a user's normal values with respect to ECG characteristics that can indicate abnormal morphologies.

The longitudinal monitoring logic 107A may first establish baseline T-wave inversion and ST depression values for the user to properly identify longitudinal changes. To do this, in some embodiments, the longitudinal monitoring logic 107A may require the user to take a predefined number of ECGs when they first begin using the interface module 101A (e.g., as part of an onboarding process) to establish a baseline median beat (not shown). The baseline median beat may be stored locally in the memory 107 as well as in the cloud storage system 113 so that it can be retrieved in the event the interface module 101A crashes or is deleted and subsequently reinstalled by the user. In other embodiments, the longitudinal monitoring logic 107A may determine the baseline median beat after the user has already been using the interface module 101A for a period of time. For example, the longitudinal monitoring logic 107A may do this by analyzing the user's existing ECG data entries stored on the local computing device 101 or in the cloud storage system 113 to establish the baseline median beat. The longitudinal monitoring logic 107A may establish the baseline median beat based on a single existing ECG data entry or multiple existing ECG data entries.

Once the baseline median beat has been established, whenever the interface module 101A stores a new ECG data entry in the memory 107, the longitudinal monitoring logic 107A may analyze the ECG data of the new ECG data entry (hereinafter referred to as “new ECG data”) by cross-correlating various waves, segments and intervals of the new ECG data with the corresponding waves, segments and intervals of the baseline median beat. Based on the determined cross-correlation of the various waves, segments and intervals of the new ECG data with the corresponding waves, segments and intervals of the baseline median beat, the longitudinal monitoring logic 107A may determine whether the new ECG data represents a deviation from the baseline median beat with respect to ECG characteristics that can indicate abnormal morphologies as well as ECG characteristics that can help to verify a source of the new ECG data. Although embodiments of the present disclosure are described with respect to the T-wave and the ST segment as ECG characteristics that can indicate abnormal morphologies and QRS complex as an ECG characteristic that can indicate a source of ECG data, this is for example purposes only and the longitudinal monitoring logic 107A may determine whether the new ECG data represents a deviation from the baseline median beat with respect to any ECG characteristics that can indicate abnormal morphologies or indicate a source of ECG data.

For example, the longitudinal monitoring logic 107A may cross-correlate the ST segment from the new ECG data with the ST segment from the baseline median beat and may cross-correlate the QRS complex from the new ECG data with the QRS complex from the baseline median beat. In another example, the longitudinal monitoring logic 107A may cross-correlate the PR segment and the TP segment from the new ECG data with the PR segment and the TP segment from the baseline median beat respectively. The longitudinal monitoring logic 107A may also cross-correlate the QRS, PR and QT intervals from the new ECG data with the QRS, PR and QT intervals from the baseline median beat respectively.

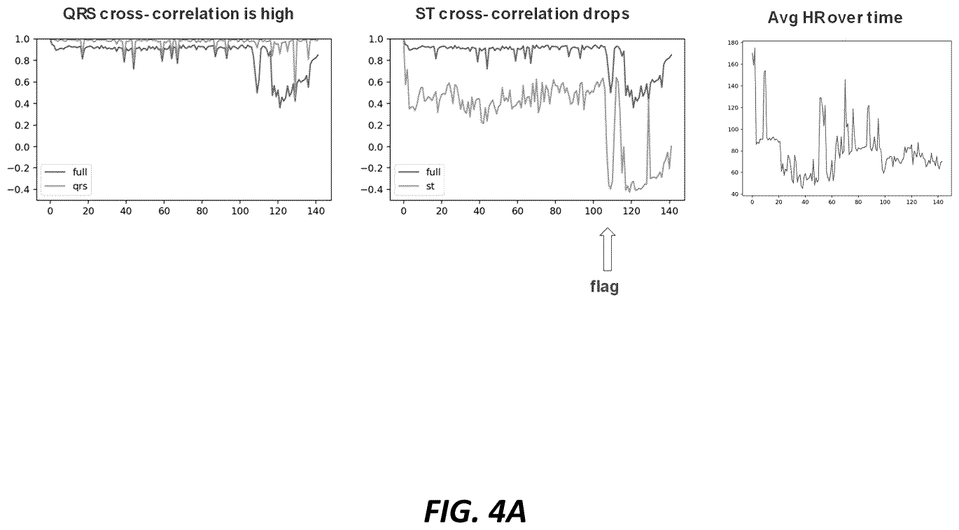

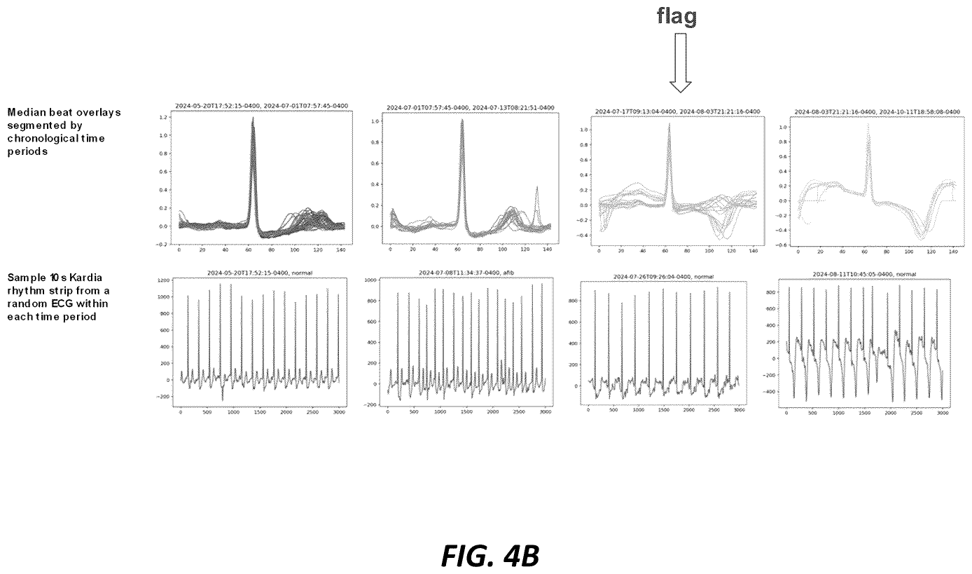

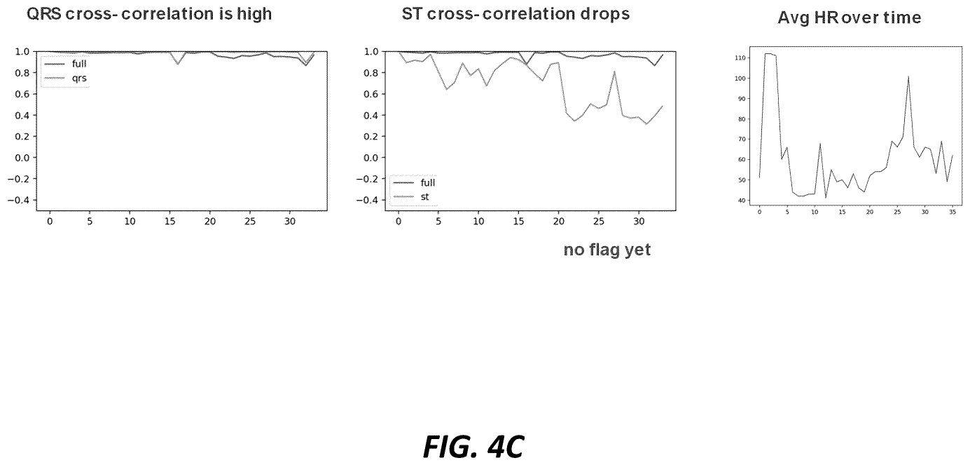

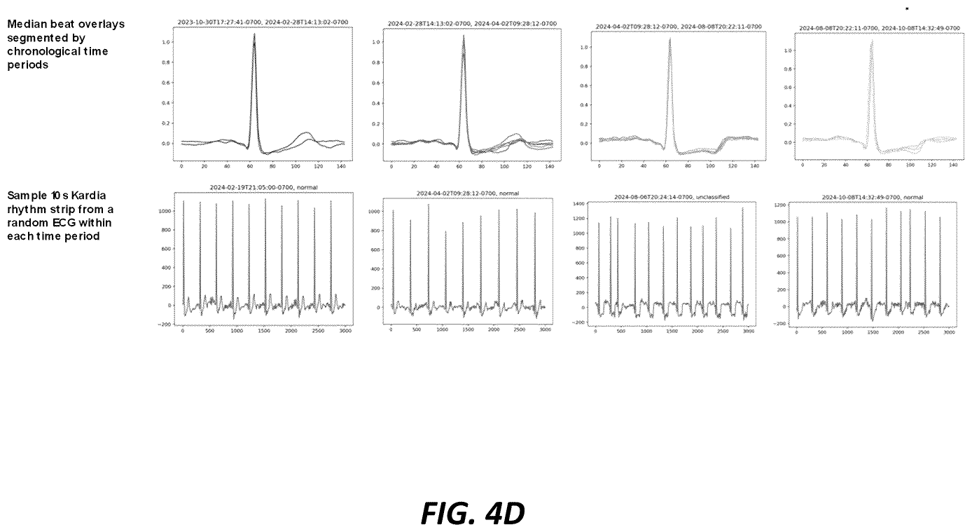

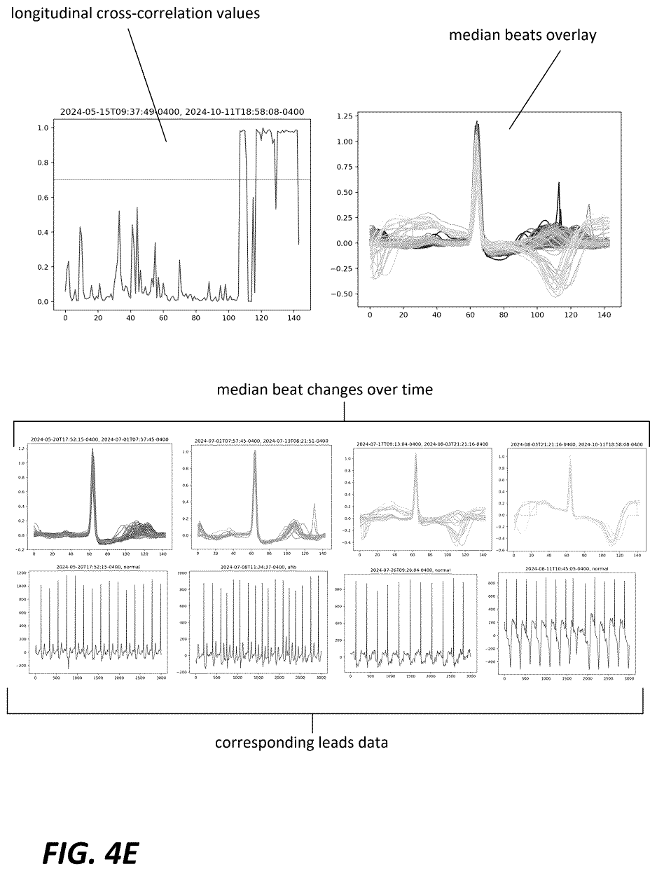

Cross-correlating various different waves, segments and intervals in this way helps to ensure that T-wave and ST segment-related morphological changes are accurately detected. Indeed, analysis based on cross-correlation of only the full new ECG data and the full baseline median beat may focus too extensively on the QRS complex cross-correlation at the cost of not providing an accurate indication of the ST segment cross-correlation (as indicated in FIG. 4A). This is undesirable because low ST segment cross-correlation likely indicates a change in ST segment and/or T-wave inflection. Similarly, a high QRS complex cross-correlation may indicate that the new ECG data likely originated from the user and not another person. Thus, the longitudinal monitoring logic 107A may analyze wave, segment and interval specific cross-correlation between the new ECG data and the baseline median beat (in addition to the cross-correlation between the full new ECG data and the full baseline median beat) to ensure that T-wave and ST segment-related morphological changes are accurately detected.

The longitudinal monitoring logic 107A may utilize threshold cross-correlation values to flag the new ECG data for a change in the ST segment and/or the T-wave or flag the new ECG data for a change in QRS complex (which may indicate the new ECG data as originating from a different user than the user associated with the baseline median beat). For example, the longitudinal monitoring logic 107A may use an ST segment cross-correlation of <=0.20 (less than or equal to 0.20), and a QRS cross-correlation of >=0.70. However, any appropriate threshold cross-correlation values may be used. Cross-correlation provides an amplitude-resistant measure of how well two independent signals resemble each other but embodiments of the present disclosure are not limited to cross-correlation and other measures of similarity between signals may be used instead of or in addition to cross-correlation.

FIG. 4A illustrates a first graph indicating the QRS complex cross-correlation (orange line) of ECG data entries for a first user versus the baseline median beat of the first user over a time period of 150 days (May 2024 to October 2024 as shown in FIG. 4A). The QRS complex cross-correlation is illustrated relative to the full cross-correlation (blue line) of the ECG data entries for the first user versus the baseline median beat of the first user over the time period (referred to herein as the full median beat cross-correlation). FIG. 4A also illustrates a second graph indicating the ST segment cross-correlation (orange line) of the ECG data entries for the first user versus the baseline median beat of the first user over the time period. The ST segment cross-correlation is illustrated relative to the full median beat cross-correlation (blue line). FIG. 4A also illustrates a graph of the first user's heart rate over time. The longitudinal monitoring logic 107A establishes the baseline median beat at day 0 and begins receiving new ECG data regularly shortly thereafter. The orange QRS complex and ST segment cross-correlation lines show why it is important to determine QRS complex and ST segment cross-correlation for each new ECG data entry to identify the relevant changes. As shown in FIG. 4A, the ST segment cross-correlation is moderately high (and maintains a generally consistent level of deviation from the full median beat cross-correlation) until approximately day 105 at which point the ST segment cross-correlation drops below 0.2 consistently (indicating a change in the ST segment) and experiences a large increase in deviation from the full median beat cross-correlation. Meanwhile, a high QRS cross-correlation is generally maintained across the entire 150-day time period and the full median beat cross-correlation tracks the QRS cross-correlation well until approximately day 105, at which point the QRS cross-correlation deviates significantly from the full median beat cross-correlation.

Analysis based only on the full median beat cross-correlation would not reveal the ST segment change or the consistency of the QRS cross-correlation after approximately day 105. Similarly, analysis based only on the cross-correlation of the QRS complex would not reveal the ST segment change, while analysis based only on the ST segment cross-correlation would not reveal the consistency or level of the QRS cross-correlation. The high cross-correlation of the QRS complex across the time period indicates that all of the ECG data entries are likely from the user. Stated differently, the QRS cross-correlation remaining high across the entire time period indicates an increased likelihood that a flagged change is due to an actual change in the ST segment and not simply a different user performing an ECG.

FIG. 4B illustrates the full median beat overlays of the ECG data entries of FIG. 4A segmented into four different chronological time periods. Each graph in the top row overlays median beats for one of the four time periods, while each graph in the bottom row provides a rhythm strip snippet of an ECG taken within one of the four time periods. FIG. 4B retrospectively indicates when the user would have been flagged as ‘Change Detected’ (using the word “FLAG” accompanied by the red arrow) using the cross correlation methodology discussed hereinabove. In the example of FIG. 4B, the user would have been flagged as ‘Change Detected’ around the time ST/T changes are noticed visually (see the top row graph corresponding to the time period from 2024July 17 to 2024 August 3 ). Corresponding changes in the rhythm can also be seen during this time. In a prospective case based on the example of FIG. 4B, healthcare provider intervention would ideally occur during this time period instead of allowing the T-wave abnormality to worsen over time without any intervention/indication.

In response to detecting that the new ECG data indicates a deviation from the baseline median beat with respect to ECG characteristics that can indicate abnormal morphologies, the longitudinal monitoring logic 107A may append a deviation indication to the corresponding new ECG data entry stored locally in memory 107 (i.e., the new ECG data entry may be flagged as ‘Change Detected’). In some embodiments, in response to identifying such a change a threshold number of times within a threshold number of new ECG data entries (e.g., 3 ECG data entries having the deviation indication within the last 5 new ECG data entries), the longitudinal monitoring logic 107A may generate an alert for the user indicating that there has been a change in the user's ECG data that requires additional analysis by a physician. In some embodiments, the longitudinal monitoring logic 107A may generate an alert for the user indicating that there has been a change in the user's ECG data that requires additional analysis by a physician in response to identifying such a change in a threshold number of consecutive new ECG data entries. Any appropriate threshold number of change identifications within (if applicable) any appropriate threshold number of new ECG data entries may be used.

In the example of FIG. 4A, the ST segment cross-correlation value drops below 0.2 consistently for 3-5 ECGs in a row, and thus the longitudinal monitoring logic 107A may generate an alert for the user indicating that there has been a change in the user's ECG data that requires additional analysis by a physician. In some embodiments, the longitudinal monitoring logic 107A may also compile a report including the ECG data entries that include the deviation indication, the corresponding dates and times of each, the amount of deviation detected in each, and other relevant information (e.g., user characteristics as described above) and send the report to a physician for an overread.

FIG. 4C illustrates graphs indicating the QRS complex cross-correlation and ST segment cross-correlation of ECG data entries for a second user versus the baseline median beat of the second user over a year long period (October 2023 to October 2024 as shown in FIG. 4C) divided into 35 time points. The longitudinal monitoring logic 107A establishes the baseline median beat at time point 0 and begins receiving new ECG data regularly shortly thereafter. The orange correlation trends show why it is important to determine QRS complex and ST segment cross-correlation for each new ECG data entry to identify the relevant changes. As shown in FIG. 4C, the ST segment cross-correlation is relatively high (~0.8) until time point 22, at which point it drops to ~0.3, increases back to ~0.8, and then drops back to ~0.3 before potentially rebounding again. Meanwhile, a high QRS cross-correlation is maintained across the entire year long time period. Although none of the ECG data entries received from time point 22 onwards are given a deviation indication (as their correlation values are still above the example 0.2 threshold), FIG. 4C shows that such ST segment changes cannot be identified based only on the cross-correlation of the QRS complex. The high cross-correlation of the QRS complex across the time period indicates that all of the ECG data entries are likely from the user. FIG. 4D illustrates the full median beat overlays of the ECG data entries of FIG. 4A segmented into different chronological time periods.

In cases where an alert indicating that there has been a change in the user's ECG data that requires additional analysis by a physician has been generated, the longitudinal monitoring logic 107A may also generate a report including the ECG data entries that include the deviation indication, the corresponding dates and times of each, the amount of deviation detected in each, and other relevant information (e.g., user characteristics as described above). The report may also include other evidence to support the overread/indication of change.

For example, in addition to longitudinal cross-correlation values, the longitudinal monitoring logic 107A may include a median beats overlay, along with median beat changes over time with corresponding leads data as evidence of a non-noise related change. In some embodiments, the interface module 101A may include a machine learning (ML) model (not shown) that is trained to detect ST segment-depression/T wave-inversion in a single ECG. The ML model's probabilities over time may also be included in the report. Other information included in the report may include the user's medical history, etc.

In some embodiments, the longitudinal monitoring logic 107A may prompt the user to confirm that they recorded the ECG where the change was detected, may prompt the user to enter any symptoms/notes that should be attached to the report, and may prompt the user to confirm they would like the report to be submitted to a physician for change detection assessment (in some embodiments, a one-time consent to send all change detection reports to a physician for overread may be obtained during the onboarding process).

FIG. 4E illustrates some of the data that can be included in the report described above for the scenario in FIG. 4A, including longitudinal cross-correlation values, a median beats overlay, median beat changes over time, and corresponding leads data which can serve as evidence of a non-noise related change. In some embodiments, some or all of the information included in the report may also be provided to the user via an interface of the interface module 101A. In some embodiments, whenever the longitudinal monitoring logic 107A completes analysis of new ECG data, it may display the results of the analysis to the user via an interface of the interface module 101A, regardless of whether a change has not been detected.

Below is a more complete (but not exhaustive) list of information that may be included in a change detection report, whether sourced from the user, a user profile, or other source:
Patient Information
Name
DOB
Age
Sex
Height
Language(s) spoken
Baseline median beat/baseline ECG
   Date ECG data in question was recorded
   Time ECG data in question was recorded
   Heart Rate when ECG data in question was recorded
   Report ID—Unique numeric identifier of ECG data
   AI interpretation (if applicable)
   Tags and Notes
ECG data in which change was detected
   Date Recorded
   Time of Recorded
   Heart Rate
   Report ID—Unique numeric identifier of EKG
   AI interpretation (if applicable)
   Tags and Notes
2 EKGs prior to the change detected EKGs
   Date Recorded
   Time of Recorded
   Heart Rate
   Report ID—Unique numeric identifier of EKG
AI interpretation (if applicable)
Median beat plot next to the change detection EKG

### Magnification of the Ekg Review Tool

In some embodiments, the interface module 101A may provide the functions of the longitudinal monitoring logic 107A to the user on an opt-out basis. For example, during an onboarding process of the interface module 101A, the interface module 101A may inquire whether the user wishes to participate in longitudinal monitoring of their ECG data.

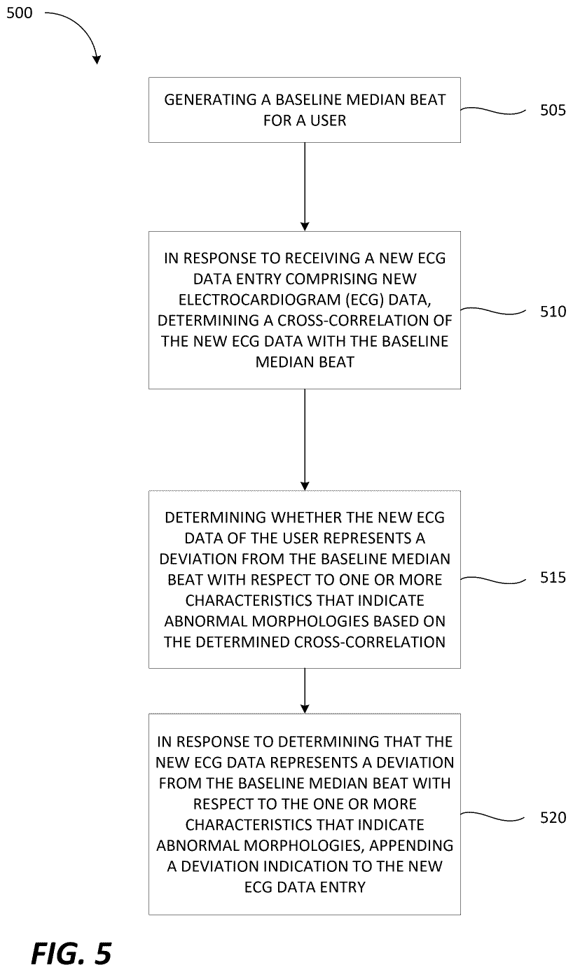

FIG. 5 is a flow diagram of a method 500 for longitudinally analyzing a user's ECG data to identify changes in the user's ECG data that could indicate the onset of cardiac conditions, in accordance with some embodiments of the present disclosure. Method 500 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 500 may be performed by a computing device (e.g., local computing device 101 illustrated in FIG. 2).

At block 505, the longitudinal monitoring logic 107A may first establish baseline T-wave inversion and ST depression values for the user to properly identify longitudinal changes. To do this, in some embodiments, the longitudinal monitoring logic 107A may require the user to take a certain number of ECGs when they first begin using the interface module 101A (e.g., as part of an onboarding process) to establish a baseline median beat (not shown). The baseline median beat may be stored locally in the memory 107 as well as in the cloud storage system 113 so that it can be retrieved in the event the interface module 101A crashes or is deleted and subsequently reinstalled by the user. In other embodiments, the longitudinal monitoring logic 107A may determine the baseline median beat after the user has already been using the interface module 101A for a period of time. For example, the longitudinal monitoring logic 107A may do this by analyzing the user's existing ECG data entries stored on the local computing device 101 or in the cloud storage system 113 to establish the baseline median beat. The longitudinal monitoring logic 107A may establish the baseline median beat based on a single existing ECG data entry or multiple existing ECG data entries.

At block 510, once the baseline median beat has been established, whenever the interface module 101A stores a new ECG data entry in the memory 107, the longitudinal monitoring logic 107A may analyze the ECG data of the new ECG data entry (hereinafter referred to as “new ECG data”) by cross-correlating various waves, segments and intervals of the new ECG data with the corresponding waves, segments and intervals of the baseline median beat. At block 515, based on the determined cross-correlation of the various waves, segments and intervals of the new ECG data with the corresponding waves, segments and intervals of the baseline median beat, the longitudinal monitoring logic 107A may determine whether the new ECG data represents a deviation from the baseline median beat with respect to ECG characteristics that can indicate abnormal morphologies as well as ECG characteristics that can help to verify a source of the new ECG data. Although embodiments of the present disclosure are described with respect to the T-wave and the ST segment as ECG characteristics that can indicate abnormal morphologies and QRS complex as an ECG characteristic that can indicate a source of ECG data, this is for example purposes only and the longitudinal monitoring logic 107A may determine whether the new ECG data represents a deviation from the baseline median beat with respect to any ECG characteristics that can indicate abnormal morphologies or indicate a source of ECG data.

For example, the longitudinal monitoring logic 107A may cross-correlate the ST segment from the new ECG data with the ST segment from the baseline median beat and may cross-correlate the QRS complex from the new ECG data with the QRS complex from the baseline median beat. In another example, the longitudinal monitoring logic 107A may cross-correlate the PR segment and the TP segment from the new ECG data with the PR segment and the TP segment from the baseline median beat respectively. The longitudinal monitoring logic 107A may also cross-correlate the QRS, PR and QT intervals from the new ECG data with the QRS, PR and QT intervals from the baseline median beat respectively.

Cross-correlating various different waves, segments and intervals in this way helps to ensure that T-wave and ST segment-related morphological changes are accurately detected. Indeed, analysis based on cross-correlation of only the full new ECG data and the full baseline median beat may focus too extensively on the QRS complex cross-correlation at the cost of not providing an accurate indication of the ST segment cross-correlation (as indicated in FIG. 4A). This is undesirable because low ST segment cross-correlation likely indicates a change in ST segment and/or T-wave inflection. Similarly, a high QRS complex cross-correlation may indicate that the new ECG data likely originated from the user and not another person. Thus, the longitudinal monitoring logic 107A may analyze wave, segment and interval specific cross-correlation between the new ECG data and the baseline median beat (in addition to the cross-correlation between the full new ECG data and the full baseline median beat) to ensure that T-wave and ST segment-related morphological changes are accurately detected.

At block 520, in response to detecting that the new ECG data indicates a deviation from the baseline median beat with respect to ECG characteristics that can indicate abnormal morphologies, the longitudinal monitoring logic 107A may append a deviation indication to the corresponding new ECG data entry stored locally in memory 107. In some embodiments, in response to identifying such a change a threshold number of times within a threshold number of new ECG data entries (e.g., 3 ECG data entries having the deviation indication within the last 5 new ECG data entries), the longitudinal monitoring logic 107A may generate an alert for the user indicating that there has been a change in the user's ECG data that requires additional analysis by a physician. In some embodiments, the longitudinal monitoring logic 107A may generate an alert for the user indicating that there has been a change in the user's ECG data that requires additional analysis by a physician in response to identifying such a change in a threshold number of consecutive new ECG data entries. Any appropriate threshold number of change identifications within (if applicable) any appropriate threshold number of new ECG data entries may be used.

In the example of FIG. 4A, the ST segment cross-correlation value drops below 0.2 consistently for 3-5 ECGs in a row, and thus the longitudinal monitoring logic 107A may generate an alert for the user indicating that there has been a change in the user's ECG data that requires additional analysis by a physician. In some embodiments, the longitudinal monitoring logic 107A may also compile a report including the ECG data entries that include the deviation indication, the corresponding dates and times of each, the amount of deviation detected in each, and other relevant information (e.g., user characteristics as described above) and send the report to a physician for an overread.

FIG. 4C illustrates graphs indicating the QRS complex cross-correlation and ST segment cross-correlation of ECG data entries for a second user versus the baseline median beat of the second user over a year long period (October 2023 to October 2024 as shown in FIG. 4C) divided into 35 time points. The longitudinal monitoring logic 107A establishes the baseline median beat at time point 0 and begins receiving new ECG data regularly shortly thereafter. The orange correlation trends show why it is important to determine QRS complex and ST segment cross-correlation for each new ECG data entry to identify the relevant changes. As shown in FIG. 4C, the ST segment cross-correlation is relatively high (~0.8) until time point 22, at which point it drops to ~0.3, increases back to ~0.8, and then drops back to ~0.3 before potentially rebounding again. Meanwhile, a high QRS cross-correlation is maintained across the entire year long time period. Although none of the ECG data entries received from time point 22 onwards are given a deviation indication (as their correlation values are still above the example 0.2 threshold), FIG. 4C shows that such ST segment changes cannot be identified based only on the cross-correlation of the QRS complex. The high cross-correlation of the QRS complex across the time period indicates that all of the ECG data entries are likely from the user. FIG. 4D illustrates the full median beat overlays of the ECG data entries of FIG. 4A segmented into different chronological time periods.

In cases where an alert indicating that there has been a change in the user's ECG data that requires additional analysis by a physician has been generated, the longitudinal monitoring logic 107A may also generate a report including the ECG data entries that include the deviation indication, the corresponding dates and times of each, the amount of deviation detected in each, and other relevant information (e.g., user characteristics as described above). The report may also include other evidence to support the overread/indication of change.

For example, in addition to longitudinal cross-correlation values, the longitudinal monitoring logic 107A may include a median beats overlay, along with median beat changes over time with corresponding leads data as evidence of a non-noise related change. In some embodiments, the interface module 101A may include a machine learning (ML) model (not shown) that is trained to detect ST segment-depression/T wave-inversion in a single ECG. The ML model's probabilities over time may also be included in the report. Other information included in the report may include the user's medical history, etc.

In some embodiments, the longitudinal monitoring logic 107A may prompt the user to confirm that they recorded the ECG where the change was detected, may prompt the user to enter any symptoms/notes that should be attached to the report, and may prompt the user to confirm they would like the report to be submitted to a physician for change detection assessment (in some embodiments, a one-time consent to send all change detection reports to a physician for overread may be obtained during the onboarding process).

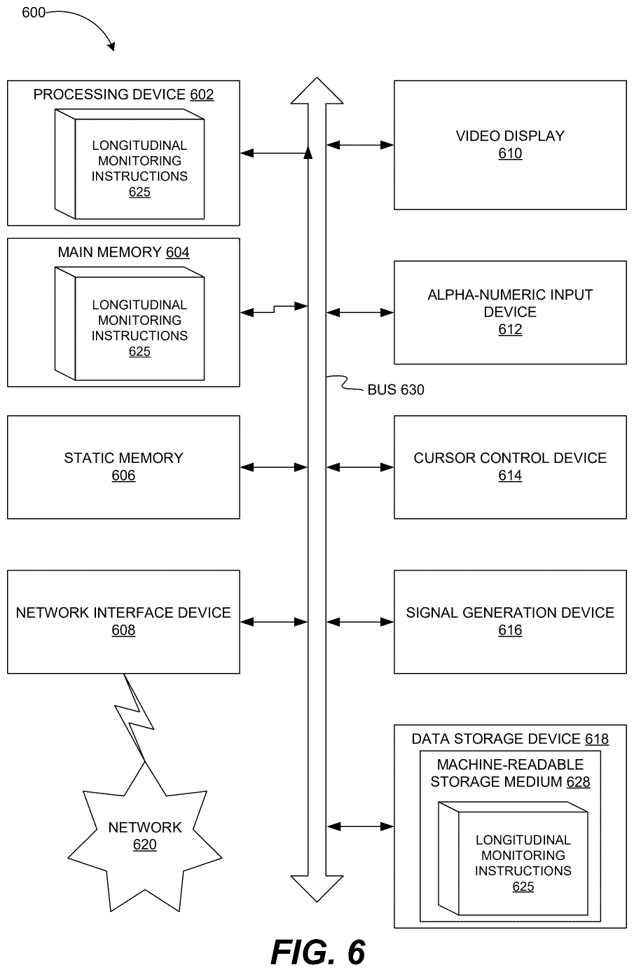

FIG. 6 illustrates a diagrammatic representation of a machine in the example form of a computer system 600 within which a set of instructions, for causing the machine to perform any one or more of the methodologies discussed herein for longitudinally analyzing a user's ECG data to identify changes in the user's ECG data that could indicate the onset of cardiac conditions.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 600 may be representative of a server.

The exemplary computer system 600 includes a processing device 602, a main memory 604 (e.g., read-only memory (ROM), flash memory, dynamic random access memory (DRAM), a static memory 606 (e.g., flash memory, static random access memory (SRAM), etc.), and a data storage device 618, which communicate with each other via a bus 630. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Computing device 600 may further include a network interface device 608 which may communicate with a network 620. The computing device 600 also may include a video display unit 610 (e.g., a liquid crystal display (LCD) or a cathode ray tube (CRT)), an alphanumeric input device 612 (e.g., a keyboard), a cursor control device 614 (e.g., a mouse) and an acoustic signal generation device 616 (e.g., a speaker). In one embodiment, video display unit 610, alphanumeric input device 612, and cursor control device 614 may be combined into a single component or device (e.g., an LCD touch screen).

Processing device 602 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. The processing device 602 is configured to execute longitudinal monitoring instructions 625, for performing the operations and steps discussed herein.

The data storage device 618 may include a machine-readable storage medium 628, on which is stored one or more sets of longitudinal monitoring instructions 625 (e.g., software) embodying any one or more of the methodologies of functions described herein. The longitudinal monitoring instructions 625 may also reside, completely or at least partially, within the main memory 604 or within the processing device 602 during execution thereof by the computer system 600; the main memory 604 and the processing device 602 also constituting machine-readable storage media. The longitudinal monitoring instructions 625 may further be transmitted or received over a network 620 via the network interface device 608.

While the machine-readable storage medium 628 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. A method comprising:
generating a baseline median beat for a user;
in response to receiving a new ECG data entry comprising new electrocardiogram (ECG) data, determining a cross-correlation of the new ECG data with the baseline median beat;
determining whether the new ECG data of the user represents a deviation from the baseline median beat with respect to one or more characteristics that indicate abnormal morphologies based on the determined cross-correlation; and
in response to determining that the new ECG data represents a deviation from the baseline median beat with respect to the one or more characteristics that indicate abnormal morphologies, appending a deviation indication to the new ECG data entry.

2. The method of claim 1, wherein determining the cross-correlation of the new ECG data with the baseline median beat comprises determining a cross-correlation between:
each of one or more waves of the new ECG data and a corresponding wave of the baseline median beat;
each of one or more segments of the new ECG data and a corresponding segment of the baseline median beat; and
each of one or more intervals of the new ECG data and a corresponding interval of the baseline median beat.

3. The method of claim 2, wherein determining whether the new ECG data of the user represents a deviation from the baseline median beat with respect to the one or more characteristics comprises:
comparing the cross-correlation determined for each of the one or more waves, each of the one or more segments and each of the one or more intervals to a corresponding threshold cross-correlation value to determine whether the new ECG data represents a deviation from the baseline median beat with respect to the one or more characteristics.

4. The method of claim 3, further comprising:
in response to the deviation indication being applied to a threshold number of consecutive new ECG data entries:
generating an alert to inform the user of a change in ECG data of the user that requires additional analysis by a physician; and
generating a report comprising:
an indication of each of the threshold number of consecutive new ECG data entries;
an amount of the deviation from the baseline median beat with respect to the one or more characteristics that indicate abnormal morphologies detected for each of the threshold number of consecutive new ECG data entries; and
an overlay of the baseline median beat indicating median beat changes over time with corresponding leads data.

5. The method of claim 4, wherein the report further includes user characteristics comprising: relevant medical history, location where the new ECG data was acquired, medication consumption, exercise patterns, sleep patterns, and activities performed before, during, or after the new ECG data was acquired.

6. The method of claim 1, further comprising:
determining, for the new ECG data, an amount of deviation from the baseline median beat with respect to QRS complex based on the determined cross-correlation; and
determining whether the new ECG data originates from the user based on the amount of deviation from the baseline median beat.

7. The method of claim 1, wherein the one or more characteristics that indicate abnormal morphologies comprise an ST segment and a T wave.

8. A system comprising:
a memory; and
a processing device operatively coupled to the memory, the processing device to:
generate a baseline median beat for a user;
in response to receiving a new ECG data entry comprising new electrocardiogram (ECG) data, determine a cross-correlation of the new ECG data with the baseline median beat;
determine whether the new ECG data of the user represents a deviation from the baseline median beat with respect to one or more characteristics that indicate abnormal morphologies based on the determined cross-correlation; and
in response to determining that the new ECG data represents a deviation from the baseline median beat with respect to the one or more characteristics that indicate abnormal morphologies, append a deviation indication to the new ECG data entry.

9. The system of claim 8, wherein to determine the cross-correlation of the new ECG data with the baseline median beat, the processing device is to determine a cross-correlation between:
each of one or more waves of the new ECG data and a corresponding wave of the baseline median beat;
each of one or more segments of the new ECG data and a corresponding segment of the baseline median beat; and
each of one or more intervals of the new ECG data and a corresponding interval of the baseline median beat.

10. The system of claim 9, wherein to determine whether the new ECG data of the user represents a deviation from the baseline median beat with respect to the one or more characteristics, the processing device is to:
compare the cross-correlation determined for each of the one or more waves, each of the one or more segments and each of the one or more intervals to a corresponding threshold cross-correlation value to determine whether the new ECG data represents a deviation from the baseline median beat with respect to the one or more characteristics.

11. The system of claim 10, wherein the processing device is further to:
in response to the deviation indication being applied to a threshold number of consecutive new ECG data entries:
generate an alert to inform the user of a change in ECG data of the user that requires additional analysis by a physician; and
generate a report comprising:
an indication of each of the threshold number of consecutive new ECG data entries;
an amount of the deviation from the baseline median beat with respect to the one or more characteristics that indicate abnormal morphologies detected for each of the threshold number of consecutive new ECG data entries; and
an overlay of the baseline median beat indicating median beat changes over time with corresponding leads data.

12. The system of claim 11, wherein the report further includes user characteristics comprising: relevant medical history, location where the new ECG data was acquired, medication consumption, exercise patterns, sleep patterns, and activities performed before, during, or after the new ECG data was acquired.

13. The system of claim 8, wherein the processing device is further to:
determine, for the new ECG data, an amount of deviation from the baseline median beat with respect to QRS complex based on the determined cross-correlation; and
determine whether the new ECG data originates from the user based on the amount of deviation from the baseline median beat.

14. The system of claim 8, wherein the one or more characteristics that indicate abnormal morphologies comprise an ST segment and a T wave.

15. A non-transitory computer-readable medium having instructions stored thereon which, when executed by a processing device, cause the processing device to:
generate a baseline median beat for a user;
in response to receiving a new ECG data entry comprising new electrocardiogram (ECG) data, determine a cross-correlation of the new ECG data with the baseline median beat;
determine whether the new ECG data of the user represents a deviation from the baseline median beat with respect to one or more characteristics that indicate abnormal morphologies based on the determined cross-correlation; and
in response to determining that the new ECG data represents a deviation from the baseline median beat with respect to the one or more characteristics that indicate abnormal morphologies, append a deviation indication to the new ECG data entry.

16. The non-transitory computer-readable medium of claim 15, wherein to determine the cross-correlation of the new ECG data with the baseline median beat, the processing device is to determine a cross-correlation between:
each of one or more waves of the new ECG data and a corresponding wave of the baseline median beat;
each of one or more segments of the new ECG data and a corresponding segment of the baseline median beat; and
each of one or more intervals of the new ECG data and a corresponding interval of the baseline median beat.

17. The non-transitory computer-readable medium of claim16, wherein to determine whether the new ECG data of the user represents a deviation from the baseline median beat with respect to the one or more characteristics, the processing device is to:
compare the cross-correlation determined for each of the one or more waves, each of the one or more segments and each of the one or more intervals to a corresponding threshold cross-correlation value to determine whether the new ECG data represents a deviation from the baseline median beat with respect to the one or more characteristics.

18. The non-transitory computer-readable medium of claim 17, wherein the processing device is further to:
in response to the deviation indication being applied to a threshold number of consecutive new ECG data entries:
generate an alert to inform the user of a change in ECG data of the user that requires additional analysis by a physician; and
generate a report comprising:
an indication of each of the threshold number of consecutive new ECG data entries;
an amount of the deviation from the baseline median beat with respect to the one or more characteristics that indicate abnormal morphologies detected for each of the threshold number of consecutive new ECG data entries; and
an overlay of the baseline median beat indicating median beat changes over time with corresponding leads data.

19. The non-transitory computer-readable medium of claim 18, wherein the report further includes user characteristics comprising: relevant medical history, location where the new ECG data was acquired, medication consumption, exercise patterns, sleep patterns, and activities performed before, during, or after the new ECG data was acquired.

20. The non-transitory computer-readable medium of claim 15, wherein the processing device is further to:
determine, for the new ECG data, an amount of deviation from the baseline median beat with respect to QRS complex based on the determined cross-correlation; and
determine whether the new ECG data originates from the user based on the amount of deviation from the baseline median beat.
